(19) United States
(12) Patent Application Publication
(10) Pub. No.: US 2005/0100594 A1
(43) Pub. Date: May 12, 2005

(54) Pharmaceutical formulation containing muscle relaxant and COX-II inhibitor

(76) Inventors: Nilendu Sen, Kopar Khairane (IN); Kavita Chandurkar, St. John's (CA); Anandi Krishnan, Vashi (IN)

Correspondence Address: Michael E. Carmen;DILWORTH & BARRESE, LLP, 333 Earle Ovington Blvd., Uniondale, NY, 11553, US

(21) Appl. No.: 10/789,054

(22) Filed: Feb. 27, 2004

(30) Foreign Application Priority Data
Nov. 12, 2003 (IN) 1180/MUM/2003

(51) Int. Cl. A61K 9/54; A61K 9/22; A61K 31/56; A61K 31/415

(52) U.S. Cl. 424/458; 514/171; 514/406; 424/468

(57) ABSTRACT

Disclosed is an extended release pharmaceutical formulation comprising a muscle relaxant drug, such as tizanidine, in combination with a cyclooxygenase-2 inhibitor, such as valdecoxib. The formulations are useful in the treatment and management of painful inflammatory conditions associated with, for example, skeletal muscle spasms.

### CLAIM FOR PRIORITY

This application claims priority from Indian provisional application number 1180/MUM/2003 filed Nov. 12, 2003. The priority application is incorporated by reference herein in its entirety.

### BACKGROUND OF THE INVENTION

1. Technical Field

The present invention relates to a pharmaceutical combination comprising a muscle relaxant drug and a cyclooxygenase inhibitor. The present invention further relates to a pharmaceutical combination of an extended release pharmaceutical formulation for oral delivery comprising a pharmaceutically effective amount of a muscle relaxant which acts as an alpha-2 adrenoreceptor agonist and an immediate release formulation of a cyclooxygenase II inhibitor useful for the treatment and management of painful inflammatory conditions associated with spasticity.

2. Description of Related Art

Traditional drug delivery systems include solid oral pharmaceutical dosage forms which are comprised of immediate release (IR) dosages in the form of tablets or capsules. These IR dosage forms release the active drug substance into the body of a subject at a rate that can initially be very high followed by a rapid decline. One potential result of an IR dosage form is that the subject may have varying degrees of blood level fluctuation, which may result in transient therapeutic overdose, followed by a period of therapeutic under dosing. These blood level fluctuations are known as “peaks” and “valleys” or “peaks” and “troughs”.

One of the most frequently utilized methods to extend the duration of drug action in the body and/or control blood level fluctuations is modification of the pharmaceutical dosage form. This is usually achieved with single or multi-component matrix systems such as, for example, granules, pellets, tablets or a combination of the above where the drug delivery is mainly controlled by a diffusion, osmotic or erosion mechanism.

Extended release formulations have the advantage that the active drug substance is gradually released over a relatively long period so that the drug is maintained in the blood stream for a longer time. The extended release formulation may also maintain the drug in the blood stream at a more uniform concentration than would otherwise be the case. This allows administration only once or twice daily for drugs that would otherwise have to be taken more frequently to maintain required blood levels. Many different types of extended release oral dosage forms have been developed, but each has disadvantages, which affect its suitability to a particular drug and therapeutic objective.

Alpha adrenoreceptor agonists play an important role in the treatment of pain by blocking nerve impulses. They may also act as skeletal muscle relaxants and can be used in combination with certain anti-inflammatory and analgesic drugs to relieve pain and also give a relaxant effect in certain arthritic conditions. Alpha adrenoreceptor agonists may be an active drug substance.

One example of a centrally acting a2 adrenergic agonist is tizanidine hydrochloride. Tizanidine HCl is a white to off-white, fine crystalline powder, odorless or with a faint characteristic odor. Tizanidine is slightly soluble in water and methanol; solubility in water decreases as the pH increases. Its chemical name is 5-chloro-4-(2-imidazolin-2-ylamino)-2,1,3-benzothiodiazole hydrochloride. Tizanidine's molecular formula is C₉H₈CIN₅S—HCl.

Tizanidine is an agonist at a2-adrenergic receptor sites and presumably reduces spasticity by increasing presynaptic inhibition of motor neurons. In animal models, tizanidine has no direct effect on skeletal muscle fibers or the neuromuscular junction, and no major effect on monosynaptic spinal reflexes. The effects of tizanidine are greatest on polysynaptic pathways. The overall effect of these actions is thought to reduce facilitation of spinal motor neurons.

The imidazoline chemical structure of tizanidine is related to that of the anti-hypertensive drug clonidine and other a2-adrenergic agonists. Pharmacological studies in animals show similarities between the two compounds, but tizanidine was found to have one-tenth to one fiftieth (1/50) of the potency of clonidine in lowering blood pressure.

Tizanidine is commercially available as 2 mg and 4 mg oral tablets under the brand name Zanaflex®. Tizanidine is administered in tablet form two to three times a day. This type of multi-dose therapy which subjects the patient to peaks and troughs has the potential for dose related side effects. One of the main side effects of Tizanidine IR tablets is sedation which may interfere with daily activity. Therefore, patients taking tizanidine should be warned about performing activities requiring alertness, such as driving a vehicle or operating heavy machinery.

One approach to improving the sedative side effects of tizanidine has been addressed in U.S. Pat. No. 6,455,557 which discloses an immediate release multiparticulate composition of tizanidine that provides a more stable serum level. However, the disadvantage of frequent dosing may still lead to reduced patient compliance.

U.S. Pat. No. 5,484,607 is directed to a controlled release system for the alpha-agonist, clonidine. The patent describes the method of preparation of an extended/sustained release matrix dosage form incorporating hydrophilic cellulose ethers as the polymeric agents for extended/sustained release of the active ingredient.

U.S. Patent Application No. 2002044966 is directed to a pharmaceutical formulation comprising in combination an opioid and an alpha-agonist wherein at least one of said opioid and alpha-agonist is present in delayed release form.

U.S. Pat. No. 4,515,802 is directed to an immediate release formulation containing a combination of an analgesic, paracetamol, and tizanidine.

International Publication No. WO 02/058620 is directed to pharmaceutical compositions and dosage forms that combine a COX-11 inhibitor and a muscle relaxant in either immediate release and/or extended release forms.

International Publication No. WO 03/005951 describes a controlled release formulation of a drug in a core, a cylindrical plug embedded in a core and a coating impermeable to the drug.

Accordingly, there remains a need to develop an extended release pharmaceutical formulation of a muscle relaxant such as tizanidine that provides once daily dosing for effective management of pain, improved side effect profile and increase patient compliance. Further, the muscle relaxant can be combined with a cyclooxygenase inhibitor, for example, valdecoxib, in an immediate release form that provides improved therapeutic response for the treatment of pain related to, for example, arthritic conditions.

### SUMMARY OF THE INVENTION

The present invention is directed towards an extended release formulation comprising at least one muscle relaxant, preferably tizanidine, which can be administered orally and releases the muscle relaxant over an extended period of time. The present invention is proposed to minimize the dose-related side effects while maintaining the efficacy and improving the patient compliance.

Controlled release has been achieved by embedding the muscle relaxant, tizanidine HCl, in a matrix of a suitable hydrophilic polymer. Controlled release can also be achieved by using controlled release coatings over the muscle relaxant, or a combination of controlled release coatings and a matrix. The formulation further contains inactive ingredients such as, for example, diluents and lubricants.

While wishing to not be bound by theory, it is believed that the hydrophilic matrix swells as soon as it comes in contact with water. The water then permeates through the swollen matrix and dissolves the drug which then diffuses out of the matrix over a period of about 14 to about 16 hours.

This formulation is advantageously combined with an anti-inflammatory drug, for example, a cyclooxygenase-2 inhibitor, and preferably, valdecoxib, in an immediate release form. Accordingly, in one embodiment of the present invention, a pharmaceutical formulation is provided comprising:
(a) a first composition comprising a therapeutically effective amount of at least one muscle relaxant; and
(b) a different second composition comprising a therapeutically effective amount of at least one cyclooxygenase-2 (COX II) inhibitor,
wherein the first composition provides a controlled and/or extended release of the muscle relaxant and the second composition provides a rapid and/or immediate release of the COX II inhibitor.

One aspect of the present invention is to provide an orally administrable dosage form that when dosed once daily to humans provides therapeutic relief from pain associated with, for example, certain arthritic conditions, by releasing the active drug substance in such a manner that requisite blood levels are maintained for periods sufficient to justify once a day dosing and thus ensure patient compliance.

Another aspect of the present invention is to provide an extended release pharmaceutical formulation that includes a core region with an effective amount of an active pharmaceutical ingredient, for example, tizanidine, which may also be advantageously combined with an immediate release formulation of a selective COX-11 inhibitor, for example, valdecoxib.

Definitions

The term “extended release” as used herein means a drug dosage system in which the rate of the drug release is more precisely controlled compared to that of immediate or sustained release products, wherein the drug is delivered from the dosage system at a predictable and predetermined rate within the body of a patient such that a therapeutically effective blood level, devoid of peak and trough fluctuations, is maintained over an extended period of time.

The term “drug delivery systems” as used herein means the technology utilized to present the drug to the desired body site for drug release and absorption.

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by local or by systemic administration of the active ingredient to the host.

By “pharmaceutically acceptable” is meant those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include the sodium, calcium, potassium and magnesium salts, and the like.

The term “subject” or “a patient” or “a host” as used herein refers to mammalian animals, preferably human.

As used herein the term “antioxidant” is intended to mean an agent who inhibits oxidation and is thus used to prevent the deterioration of preparations by the oxidative process. Such compounds include, by way of example and without limitation, ascorbic acid, ascorbic palmitate, Vitamin E, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisulfite and other such materials known to those of ordinary skill in the art.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly(vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68 and PLURONIC™ f127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

As used herein, the term “glidant” is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

As used herein, the term “lubricant” is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

Most of these excipients are described in detail in Howard C. Ansel et al., Pharmaceutical Dosage Forms and Drug Delivery Systems, (7th Ed. 1999); Alfonso R. Gennaro et al., Remington. The Science and Practice of Pharmacy, (20th Ed. 2000); and A. Kibbe, Handbook of Pharmaceutical Excipients (3^{rd} Ed. 2000), which are incorporated by reference herein.

### DETAILED DESCRIPTION OF THE PREFERRED EMBODIMENTS

The present invention relates to extended release pharmaceutical formulations for the management of pain and spacticity related to, for example, skeletal muscle spasms. It has been found possible to formulate a tablet composition of a muscle relaxant, e.g., an alpha-2 adrenergic agonist such as, for example, tizanidine, where the muscle relaxant is released in an extended release manner and a cyclooxygenase-2 inhibitor in immediate release form. The present invention provides a composition giving a plasma-concentration profile that proposes to minimize the dose-related side-effects of the drug while maintaining the efficacy within the therapeutic index.

There are several ways of achieving extended release of the muscle relaxant herein. It may be achieved by a controlled release coating, embedding the drug in a controlled release matrix or a combination thereof. Suitable muscle relaxants for use in the pharmaceutical formulation herein include those disclosed in Remington's Pharmaceutical Sciences, 16th Ed., 1980, Mack Publishing Co., Easton, Pa. and in Goodman and Gilman's The Pharmacological Basis of Therapeutics by Hardman and Limbird, 9th Ed., 1996, McGraw-Hill, N.Y., the contents of which are incorporated by reference herein. Examples include, but are not limited to, neuromuscular blocking drugs, e.g., dantrolene sodium, gallamine triethiode, hexafluorenium bromide, metocurine iodide, pancuronium bromide, succinylcholine chloride, tubocurarine chloride, curare, atracurium besylate and the like and mixtures thereof; centrally acting muscle relaxants, e.g., baclofen, carisoprodol, chlorzoxazone, cyclobenzaprine hydrochloride, methocarbamol, chlorphenesin, metaxalone, and the like and mixtures thereof; antiparkinson drugs, e.g., amantadine hydrochloride, benztropine mesylate, biperiden, biperiden hydrochloride, bromocriptine mesylate, carbidopa, levodopa, trihexyphenidyl hydrochloride, phenelzine sulfate, chlorphenoxamine hydrochloride, cycrimine hydrochloride, ethopropazine hydrochloride, orphenadrine citrate, pergolide mesylate, procyclidine hydrochloride and the like and mixtures thereof; alpha-2 adrenergic agonists, e.g., clonidine, tizanidine and the like and mixtures thereof.

In one embodiment of the present invention, the muscle relaxant is provided in an extended release formulation, where the extended release is controlled by a controlled release coating. The formulation includes a core region, which may be, for example, irregular shaped or a collection of granules. The active drug substance is included in the core region. There may also be other pharmaceutically active ingredients or pharmaceutically acceptable excipients present in the core region. In order to achieve a controlled release coating, the core region can be coated as a whole, or the core region may consist of granules and given varying coats. The varying coats may be of different thicknesses, while some of the active substance, preferably less than twenty five percent, may be completely uncoated for immediate release. The granules may then be mixed to achieve a blend of granules with varying coatings. Also, the material of the coating may be varied to achieve a controlled release coating, or a combination of varying coating thicknesses and coating materials may be used. Suitable controlled release coatings include, for example, water insoluble waxes, water insoluble polymers, for example, acrylic resins, and water insoluble celluloses, for example, ethyl cellulose.

In one preferred embodiment of the present invention, the controlled release coating is poly(meth)acrylate, an acrylic resin. Eudragit® RSPO and Eudragit® NE 30D, available from BASF® of Ludwigshafen, Germany, belong to the class of methacrylic acid copolymer namely poly(meth)acrylates copolymers. Poly(meth)acrylates are synthetic cationic and anionic polymers of dimethylaminoethylmethacrylates, methacrylic acid and methacrylic acid esters in varying ratios. They are anionic in character, based on methacrylic acid and methyl methacrylate, for example, having a ratio of free carboxyl groups to methyl-esterified carboxyl groups of about 1:<3, e.g. around about 1:1 or about 1:2, and with a mean molecular weight of about 135,000. They have solubility in aqueous media at about pH 5.5. Such polymers may be used either alone or with a plasticizer. They are used to form water insoluble film coats for sustained release products, for example, controlled release coating.

Eudragit® RS PO is a fine white powder with a slight amine-like odor. It is characteristically the same polymer as Eudragit® RL and RS. It contains greater than 97% of dry polymer.

Eudragit® NE 30 D is an aqueous dispersion of a neutral copolymer consisting of polymethacrylic acid esters. The dispersions are milky-white liquids of low viscosity and have a weak aromatic odor. Films prepared from the lacquer swell in water, to which they become permeable. Thus, films produced are insoluble in water, but give pH-independent drug release.

Polymethacrylate copolymers are widely used as film-coating materials in oral pharmaceutical formulations. Larger quantities of about 5 to about 20% of dry polymer are used to control the release of an active substance from a tablet matrix, see, for example, Handbook of Pharmaceutical Excipients—Third Edition by Raymond C. Rowe et al., which is hereby incorporated by reference. They are generally regarded as nontoxic and nonirritant materials. A daily intake of about 2 mg/kg body weight of Eudragit® (equivalent to approximately 150 mg for an average adult) may be regarded as essentially safe in humans.

Ethyl cellulose dispersion (available as Surelease® from Colorcon® of West Point, Pa.) is a unique combination of film-forming polymer, plasticizer and stabilizer. Designed for sustained release and taste masking applications, Surelease® is an easy to use, substantially aqueous coating system using ethyl cellulose as the release rate controlling polymer. The dispersion provides the flexibility to adjust drug release rates with reproducible profiles that are relatively insensitive to pH. The principal means of drug release is by diffusion through the ethyl cellulose membrane and is directly controlled by film thickness. Increasing or decreasing the quantity of Surelease® applied can easily modify the rate of release. Usually, ethyl cellulose is used in the form of sustained release coating in the concentration of about 3 to about 20%. But it may also be used as a filler in the matrix to sustain drug release. With Surelease® dispersion, reproducible drug release profiles are consistent right through development to scale-up and production processes. Its benefits are that it is a—substantially aqueous system and a ready plasticized formulation. It gives consistent, uniform drug release independent of pH. The release rates produced are reproducible through scale-up.

In order to regulate the rate of release of the active substance, the controlled release coating may also contain, in addition to the water insoluble polymers, water soluble polymers that act as channeling agents, for example, polyvinylpyrrolidone, water soluble celluloses, for example, hydroxypropyl methylcellulose or hydroxypropylcellulose, or hydrophilic pore formers, for example sucrose, sodium chloride or mannitol and/or known plasticizers.

In another embodiment of the present invention, the muscle relaxant is provided in an extended release formulation, where the presence of the active drug substance in the matrix controls the extended release formulation. The active drug substance is integrated into the matrix. The active drug substance may be in granular form and mixed with a matrix material. In use, the matrix material slowly erodes in body fluids, thereby releasing the active drug substance over a period of time. These granules may then be mixed with other active drug substance granules that have not been mixed with a matrix material. The granules not mixed with a matrix material will provide immediate release. In the present invention, the active substance will preferably be uniformly distributed in the matrix.

Physiologically compatible, hydrophilic materials, which are known to persons skilled in the art, may be used as matrix materials. Hydrophilic matrix materials which are used are polymers, preferably cellulose ethers, cellulose esters and/or acrylic resins. Especially preferred matrix materials include ethyl cellulose, hydroxypropyl methylcellulose, hydroxypropylcellulose, hydroxymethylcellulose, poly(meth)acrylic acid and/or the derivatives thereof, such as the salts, amides or esters. Hydrophobic materials, such as hydrophobic polymers, waxes, fats, long-chain fatty acids, fatty alcohols or corresponding esters or ethers or mixtures thereof can also be used as matrix materials. It is also possible to use mixtures of hydrophilic and hydrophobic materials as a controlled release matrix material.

In a preferred embodiment of the invention, the matrix is selected from the group consisting of hydroxypropyl methylcelluloses (HPMC) HPMC K15M, HPMC K100M, HPMC K100M CR (available as Methocel® and Ethocele from Dow of Midland, Mich.). Primary control of drug release is achieved by the Methocel® content, varying the ratio of drug to polymer. As the proportion of hydroxypropyl methylcellulose increases, the release rate is reduced. In the case of less water soluble drugs, viscosity type offers a secondary control mechanism.

Hydroxypropyl methylcellulose is a very versatile material for the formulation of soluble matrix tablets. HPMC is a widely accepted pharmaceutical excipient and is available in a wide range of molecular weights. Effective control of gel viscosity is easily provided. Hydroxypropyl methylcellulose is primarily used as a tablet binder in film coatings and as an extended release tablet matrix. Concentrations of between about 2 and about 5% w/w may be used as a binder in either wet or dry granulation processes. High viscosity grades may be used to retard the release of drugs from a matrix at levels of about 10 to about 80% w/w in tablets.

Polyethylene oxide polymers (available as Polyox® WSR 301 from Dow of Midland, Mich.), according to the present invention, are non-ionic, high molecular weight water soluble polymers. Molecular weights ranging from about 100,000 to about 8,000,000 can be employed herein and meet the requirements of the Food Chemicals Codex, the International Codex Alimentarius and US Pharmacopoeia (USP) or National Formulary (NF). These polymers are white, free-flowing hydrophilic powders with a long history of successful applications in pharmaceutical products, in uses such as controlled release solid dose matrix systems. The higher molecular weight grades provide delayed drug release via the hydrophilic matrix approach. Polyox® resins are used as controlled release solid dose matrix systems in the concentrations of about 1 to about 5%. Polyox® resins are very versatile polymers for controlled release applications. Upon exposure to water or gastric juices, they hydrate and swell rapidly to form hydrogels with properties ideally suited for controlled drug-delivery vehicles. No interaction between drug and polymer is to be expected because Polyox®resins are nonionic.

Several factors affect the rate of drug release from an extended release polymeric matrix. The physicochemical characteristics of the drug such as degree of water solubility, molecular weight and the diffusion coefficient from the hydrated matrix play a very important role in determining the mechanism of drug release. Also, physicochemical characteristics of the diluents added to the matrix affect the rate of drug release.

In another embodiment of the present invention, the muscle relaxant is provided in an extended release formulation, where the extended release is controlled by both the presence of the active drug substance in the matrix and a controlled release coating surrounding the core region.

Tablet excipients, as per the present invention, may be, for example, diluents, retardants, and lubricants, as well as other pharmaceutically acceptable excipients. These excipients may be present in the core region and/or any other region of the formulation. Diluents according to the present invention are inert materials needed to be added to the active ingredient to make them more acceptable. Diluents are fillers designed to make up the required bulk of the tablet when the drug dosage itself is inadequate to produce this bulk. Tablet formulations may contain diluents for secondary reasons, such as to provide better tablet properties such as improved cohesion, to permit use of direct compression manufacturing, or to promote flow.

Lactose is the most widely used diluent in tablet formulation. Lactose is an excipient that has no reaction with most drugs, whether it is used in the hydrous or anhydrous form. Anhydrous lactose has the advantage over lactose in that it does not undergo the Maillard reaction, which can lead to browning and discoloration with certain drugs. Lactose formulations show good drug release rates, their granulations are readily dried and the tablet disintegration times of lactose tablets are not strongly sensitive to variations in tablet hardness. Lactose is also a low cost diluent. Usually fine grades of lactose are used in the preparation of tablets because the fine size permits better mixing with other formulation ingredients and utilizes the binder more efficiently. Generally, the grade of lactose chosen is dependent on the type of dosage form being developed. Direct-compression grades are often used to carry small quantities of drug and this permits tablets to be made without granulating.

Direct-compression grades of lactose are more fluid and more compressible than crystalline or powdered lactose, and generally, they are composed of spray-dried lactoses, which contain specially prepared pure alpha lactose monohydrate along with a small amount of amorphous lactose. The amorphous lactose improves the compression force/hardness profile of the lactose. Other specially produced direct-compression grades of lactose do not contain amorphous material but may contain glassy or vitreous areas which impart improved compressibility. The use of direct-compression grades of lactose results in tablets of higher breaking strength than standard lactose. Concentrations of lactose generally used in these formulations are from about 65 to about 85%.

Anhydrous dibasic calcium phosphate is used in pharmaceutical products because of its compaction properties, and the good-flow properties of the coarse grade material. The predominant deformation mechanism of anhydrous dibasic calcium phosphate coarse-grade is brittle fracture and this reduces the strain sensitivity of the material, thus allowing easier transition from the laboratory to production scale. Anhydrous dibasic calcium phosphate is abrasive and a lubricant is required for tabletting, for example, about 1% magnesium stearate or about 1% sodium stearyl fumarate. Two particle size grades of anhydrous dibasic calcium phosphate are used in the pharmaceutical industry. Milled material is typically used in wet-granulated or roller-compacted formulations. The unmilled or coarse-grade material is typically used in direct-compression formulations. Anhydrous dibasic calcium phosphate is non-hygroscopic and stable at room temperature. It does not hydrate to form the dihydrate.

Retardants control the release of an active substance from a tablet matrix. As mentioned above, retardation of drug release may be achieved either by a controlled release coating or by embedding the drug in a controlled release matrix of a hydrophilic or hydrophobic polymer or a combination thereof.

Pregelatinized starch (available as Starch 1500® from Colorcon® of West Point, Pa.), according to the present invention, influences the drug release from hydroxypropyl methylcellulose sustained release matrix formulations. Use of Starch 1500® significantly reduces the drug release as compared to formulations containing MCC or lactose. Starch 1500® is not an inert filler in HPMC matrices, but it actively contributes to the mechanism of drug release causing a decrease in drug release rate. Increasing concentrations of Starch 1500® (about 20, about 35 and about 49.25% w/w) in the formulations caused a decrease in their release profiles. Drug release from matrices containing Starch 1500® was slower than when lactose or MCC was used.

In a preferred embodiment of the present invention, the extended release pharmaceutical formulation of tizanidine is uniformly dispersed in a matrix comprised of hydroxypropyl methyl cellulose and partially or fully pregelatinized starch.

Microcrystalline cellulose (available as Avicel® from FMC Corporation of Philadelphia, Pa.), according to the present invention, is a direct compression material. It is the most compactable material available for pharmaceutical use. The flow properties of the material are generally good, and the direct compression characteristics are excellent. This is a somewhat unique diluent in that while producing cohesive compacts, the material also acts as a disintegrating agent. Due to the self disintegrating property of Avicel® it requires little lubricant. Microcrystalline cellulose is often added to tablet formulation for several possible functions. It is a commonly employed excipient. The present invention employs Avicel® pH-102 in the concentration of about 20 to about 90%. In addition to its use as a binder/diluent, microcrystalline cellulose also has some lubricant properties.

Lubricants reduce friction by interposing a film of low shear strength between the tablet mass and the confining die wall interface during tablet formation and ejection. They also play the role of anti-adherents wherein they prevent sticking to surfaces like the faces of tablet punches. Lubricants also act as glidants thereby improving the flow by modifying the interaction between particles. Therefore the concept of a lubricant system is generally the use of two substances to maximize overall lubricant effect in all three areas as lubricant, antiadherent and glidant, for example, combining magnesium stearate with colloidal silica.

Stearic acid, according to the present invention, acts as a lubricant. Stearic acid acts as a lubricant in the concentration of about 1 to about 3%.

Colloidal silicon dioxide (available as Aerosil® from Degussa AG of Dusseldorf, Germany), according to the present invention, is widely used in pharmaceuticals especially in tablets as a glidant. Aerosil® acts as a glidant in the concentration of about 0.1 to about 0.5%. Its small particle size and large specific surface area give it desirable flow characteristics which are exploited to improve the flow properties of dry powders in tabletting.

The pharmaceutical formulation of the present invention may contain other optional ingredients that are also typically used in pharmaceuticals such as, for example, coloring agents, preservatives, flavorings, and the like.

In a particularly preferred embodiment of the present invention, an extended release pharmaceutical formulation includes a muscle relaxant and a COX-11 inhibitor. Preferably, the muscle relaxant is in an extended release component of the formulation, and the COX-11 inhibitor is in an immediate release component of the formulation. The muscle relaxant may be, for example, tizanidine. The COX-II inhibitor may be, for example, valdecoxib. The COX-II inhibitor may be, for example, a layer on top of the core region or part of a bilayered tablet.

Any selective COX-11 inhibitor may be used in the formulations and methods of the present invention. Useful COX-11 inhibitors that can be used in this invention include, but are not limited to, those disclosed in U.S. Pat. Nos. 5,393,790; 5,418,254; 5,420,343; 5,466,823; 5,476,944; 5,486,534; 5,547,975; 5,565,482; 5,576,339; 5,580,985, 5,585,504; 5,593,994 and 5,596,008, the contents of which are incorporated by reference herein. More particularly, the useful COX-11 inhibitors include the substituted spiro compounds of U.S. Pat. No. 5,393,790, e.g., 5-(4-fluorophenyl)-6-[4-(methylsulfonyl)phenyl]spiro[2.4]hept-5-ene, 4-[6-(4-fluorophenyl)spiro[2.4]hept-5-en-5-yl]benzenesulfonamide, 6-(4-fluorophenyl)-7-[4-(methylsulfonyl)phenyl]spiro[3.4]oct-6-ene, and the like; the sulfonamides of U.S. Pat. No. 5,409,944, e.g., 5-methanesulfonamido-6-(2-thienylthio)-1-indanone, 5-methanesulfonamido-6-(2-(4-methyl-1,3-diazinylthio))-1-indanone, 5-methanesulfonamido-6-(2-thiazolylthio)-1-indanone, and the like; the 2,3-substituted cyclopentadienyl compounds of U.S. Pat. No. 5,418,254, e.g., 1-methylsulfonyl-4-[1,1-dimethyl-4-(4-fluorophenyl)cyclopenta-2,4-dien-3-yl]benzene, 4-[4-(4-fluorophenyl)-1,1-dimethylcyclopenta-2,4-dien-3-yl]benzenesulfonamide, 1-methylsulfonyl-4-{4-(4-trifluoromethylphenyl)-1-trifluoromethylcyclopenta-2,4-dien-3-yl]benzene, and the like; the aromatic cycloethers of U.S. Pat. No. 5,420,343, e.g., methyl 3,5-bis(1,1-dimethylethyl)benzoate, 3,5-bis(1,1-dimethylethyl)benzenemethanol, 1,3-bis(1,1-dimethylethyl)-5-(2-chloroethyl)benzene, and the like; the 1-aroyl acids of U.S. Pat. No. 5,436,265, e.g., 1-(2,4,6-trichlorobenzoyl)-5-methoxy-2-methyl-3-indolyl acetic acid, 1-(2,6-dichlorobenzoyl)-5-methoxy-2-methyl-3-indolyl acetic acid and the like; the phenyl heterocycles of U.S. Pat. Nos. 5,474,995, 5,536,752, 5,550,142, 5,710,140 and 5,767,291, e.g., 3-(4-(aminosulfonyl)phenyl)-2-(4-fluorophenyl)thiophene, 2-(4-fluorophenyl)-3-(4-(methylsulfonyl)phenyl)-2-cyclopentenone, 4-(4-methylsulfonyl)phenyl)-5-(4-fluorophenyl)isothiazole, 4-(aminosulfonyl)phenyl)-2-(4-fluorophenyl)thiophene, 3-(4-(aminosulfonyl)phenyl)-2-4-(fluorophenyl)-5-(2-propyl)thiophene, 3-(4-(aminosulfonyl)phenyl)-2-cyclohexylthiophene, 3-(4-(aminosulfonyl)phenyl)-2-4-(fluorophenyl)-5-(2-hydroxy-2-propyl)thiophene, 3-(4-(aminosulfonyl)phenyl)-2-(4-fluorophenyl)thiophene and the like; the benzenesulfonamides of U.S. Pat. No. 5,466,823, e.g., 4-(5-(4-methylphenyl)-3-(trifluoromethyl)-1H-pyrazol-1-yl)benzenesulfonamide (which is also referred to as celecoxib) and the like; the cyclic phenolic thioether derivatives of U.S. Pat. No. 5,476,944, e.g., 3,5-bis(1,1-dimethylethyl)benzenethiol, trans-2-[[3,5-bis(1,1-dimethylethyl)henyl]hio]cyclohexanol, 3,6-dioxabicyclo-[3.1.0]hexane, and the like; the 3,4-substituted pyrazoles of U.S. Pat. No. 5,486,534, e.g., 4-(4-fluorophenyl)-1-methyl-3-[4-(methylsulfonyl)phenyl]-5-trifluoromethyl)pyrazole, 1-benzyl-4-(4-fluorophenyl)-3-[4-(methylsulfonyl)phenyl]-5-(trifluoromethyl)pyrazole, 1-allyl-4-(4-fluorophenyl)-3-[4-methylsulfonyl)phenyl]-5-(trifluoromethyl)-1H-pyrazole, and the like; the N-benzyl-3-indoleacetic acids of U.S. Pat. No. 5,510,368, e.g., 2-(5-bromo-1-(4-bromobenzyl)-2-methyl-1H-indol-3-yl)propionic acid, (S)-(+)-2-(5-bromo-1-(4-bromophenyl)-2-methyl-1H-indol-3-yl)acetyl acid, (R)-(−)-2-(5-bromo-1-(4-bromobenzyl)-2-methyl-1H-indol-3-yl)propionic acid, and the like; the diaryl bicyclic heterocyclics of U.S. Pat. No. 5,521,213, e.g., 3-(4-(methylsulfonyl)phenyl)-2-phenylbenzo[b]furan, 3-(4-(methanesulfonyl)phenyl)-2-phenylbenzo[b]thiophene, 2-(4-fluorophenyl)-3-(4-aminosulfonyl)phenyl)-4H-thieno[2,3-c]furan-6-one, and the like; the benzopyranopyrazolyl derivatives of U.S. Pat. No. 5,547,975, e.g., 4-[1,4-dihydro-3-(trifluoromethyl)-[1]benzopyrano[4,3-c]pyrazol-1-yl]benzenesulfonamide, methyl[1-[4-(aminosulfonyl)phenyl]-1,4-dihydro-[1]benzopyrano [4,3-c]pyrazol-3-yl]carboxylate, 4-[3-(trifluoromethyl)-1H-benzofuro[3,2-c]pyrazol-1-yl]benzenesulfonamide, and the like; the aryl substituted 5,5 fused aromatic nitrogen compounds of U.S. Pat. No. 5,552,422, e.g., 5-(4-methylsulfonyl)phenyl)-6-phenylimidazo[2,1-b]thiazole, 2-methyl-5-(methylsulfonyl)phenyl)-6-phenylimidazo[2,1-b]thiazole, 3-methyl-5-(4-methylsulfonyl)phenyl)-6-phenylimidazo[2,1-b]thiazole, and the like; the heteroarylpyranopyrazolyl derivatives of U.S. Pat. No. 5,565,482, e.g., 4-[1,5-dihydro-6-fluoro-7-methoxy-3-(trifluoromethyl)-[2]benzothiopyrano[4,3-c]pyrazol, 4-[1,4-dihydro-3-(trifluoromethyl)-[1]benzopyrano-[4,3-c]pyrazol-1-yl]benzenesulfonamide, 1,5-dihydro-6-fluoro-7-methoxy-1-[(4-methylsulfonyl)phenyl]-3-(trifluoromethyl)-[2]benzothiopyrano-[4,3-c]pyrazol-1-yl]benzenesulfonamide, and the like; the pyridyl substituted cyclopentadienes of U.S. Pat. No. 5,576,339, e.g., 1-methylsulfonyl-4-[1,1-dimethyl-4-(4-fluorophenyl)cyclopenta-2,4-dien-3-yl]benzene, 4-[4-(4-fluoropyhenyl)-1,1-dimethylcyclopenta-2,4-dien-3-yl]benzenesulfonamide, and the like; the substituted pyrazoles of U.S. Pat. No. 5,580,985, e.g., 1-ethyl-4-(4-fluorophenyl)-3-[4-(methylsulfonyl)phenyl]-5-(trifluoromethyl)-1H-pyrazole, 3-amino-4,4,4-trifluoro-2(4-fluorophenyl)-1-[4-(methylthio)phenyl]-2-buten-1-one, 1-benzyl-4-(4-fluorophenyl)-3-[4-(methylsulfonyl)phenyl]-5-(trifluoromethyl)pyrazole, and the like; the lactones of U.S. Pat. No. 5,585,504, e.g., 3-phenyl-4-(4-methylsulfonyl)phenyl-2-(5H)-furanone, 3-(3,4-difluorophenyl)-4-(4-methylsulfonyl)phenyl)-2-(5H)-furanone, and the like; the ortho substituted phenyl compounds of U.S. Pat. No. 5,593,994, e.g., 2-[(4-methylthio)phenyl]-1-biphenyl,1-cyclohexene-2-(4′-methylsulfonylphenyl)benzene, 3-(4′-methylsulfonylphenyl)-4-phenylphenol, and the like; the 3,4-diaryl substituted pyridines of U.S. Pat. No. 5,596,008, e.g., 5-(4-fluorophenyl)-2-methoxy-4-[4-(methylsulfonyl)phenyl]-6-(trifluoromethyl)pyridine, 2-ethoxy-5-(4-fluorophenyl)-4-[4-(methylsulfonyl)phenyl]-6-(trifluoromethyl)pyridine, 5-(4-fluorophenyl)-4-[4-(methylsulfonyl)phenyl]-2-(2-propynyloxy)-6-(trifluoromethyl)pyridine, and the like; the N-benzylindol-3-yl propanoic acid derivatives of U.S. Pat. No. 5,604,253, e.g., 3-[1-(p-Bromobenzyl)-5-methoxy-2-methylindol-3-yl]propanoic acid, 3-[1-(p-Bromobenzyl)-5-methoxy-2-methylindol-3-yl]-2,2-dimethyl-propanoic acid, 2-Benzyl-3-[1-(p-Bromobenzyl)-5-methoxy-2-methylindol-3-yl]propanoic acid and the like; the 5-methanesulfonamido-1-indanones of U.S. Pat. No. 5,604,260, e.g., 4-(2,4-Dichlorophenoxy)-3-nitrobenzaldehyde, 5-methanesulfonamido-6-(2,4-difluorophenylthio)-1-indanone and the like; the N-benzylindol-3-yl butanoic acid derivatives of U.S. Pat. No. 5,639,780, e.g., [4-(1-(4-Bromobenzyl)-5-methoxy-2-methyl-1-H-indol-3-yl)-3-(ethane-1,2-diyl)]butanoic acid, 4-(1-(4-Bromobenzyl)-5-methoxy-2-methyl-1-H-indol-3-yl)-2-methylbutanoic acid and the like; the diphenyl-1,2-3-thiadiazoles of U.S. Pat. No. 5,677,318, e.g., 4-Phenyl-5-(4-(methylsulfonyl)-phenyl-1,2,3-thiadiazole, 4-(4-fluorophenyl)-5-(4-(methylsulfonyl)phenyl-1,2,3-thiadiazole, 4-(3-fluorophenyl)-5-(4-(methylsulfonyl)phenyl-1,2,3-thiadiazole and the like; the diaryl-5-oxygenated-2-(5H)-furanones of U.S. Pat. No. 5,691,374, e.g., 5-hydroxy-3-(3,4-difluorophenyl)-4-(4-(methylsulfonyl)phenyl)-2-(5H)-furanone, 5-hydroxy-5-methyl-,4-(4-(methylsulfonyl)phenyl)-3-phenyl-2-(5H)-furanone, 5-hydroxy-4-(4-(methylsulfonyl)phenyl)-3-phenyl-2-(5H)-furanone and the like; the 3,4-diaryl-2-hydroxy-2,5-dihydrofuranes of U.S. Pat. No. 5,698,584, e.g., 3-(3,5-difluorophenyl)-5,5-dimethyl-2-hydroxy-4-(4-(methylsulfonyl)phenyl)-2,5-dihydrofuran, 5,5-dimethyl-3-(4-fluorophenyl)-2-hydroxy-4-(4-(methylsulfonyl)phenyl)-2,5-dihydrofuran, 5,5-dimethyl-2-ethoxy-3-(3-fluorophenyl)-4-(4-(methylsulfonyl)phenyl)-2,5-dihydrofuran and the like; the diphenyl stilbenes of U.S. Pat. No. 5,733,909, e.g., (E)-3-(4-methylsulfonyl)phenyl-2-phenylbut-2-enoic acid methyl ester, (E)-3-(methylsulfonyl)phenyl-2-phenylbut-2-enoic acid, (E)-3-(4-methylsulfonyl)phenyl-1-morpholin-4-yl-2-phenylbut-2en-1-one and the like; the alkylated styrenes of U.S. Pat. No. 5,789,413, e.g., 2-(3-fluorophenyl)-4-methyl-3-(4-(methylsulfonyl)-phenyl)-2-(Z)-penten-1.4-diol, acetic acid 4-acetoxy-2-(3-fluorophenyl)-4-methyl-3-(4-(methylsulfonyl)phenyl)-2-(Z)-pent-2-enyl ester, 2-(3-fluorophenyl)-4-methoxy-4-methyl-3-((4-methylsulfonyl1phenyl)-2-(Z)-pentenoic acid and the like; the bisaryl cyclobutene derivatives of U.S. Pat. No. 5,817,700, e.g., 4,4-dichloro-3-(4-methylthiophenyl)-2-phenyl-2-cyclobuten-1-one, 4,4-dichloro-3-(4-methylsulfonylphenyl)-2-phenyl-2-cyclobuten-1-one, 4-chloro-3-(4-methylsulfonylphenyl)-2-phenyl-2-cyclobuten-1-one and the like, the contents of each of which are incorporated by reference herein. Other selective inhibitors of COX-11 and methods of preparation thereof are set forth in, for example, WO99/30721, the contents of which are incorporated herein by reference.

Preferred selective inhibitors of COX-11 for use herein include valdecoxib, celicoxib, paracoxib, etoricoxib, 4-[5-(4-methylphenyl)-3-(triluoromethyl)-1H-pyrazol-1-yl]benzenes-sulfonamide which is compound (4) of WO99/30721 and is denoted celicoxib (trade name Celebrex®) and 3-(phenyl)-4-(4-(methylsulfonyl)phenyl)-2—(5H)-furanone which is compound (63) of WO99/30721 and is denoted MK-0966 and Vioxx®. Another selective inhibitor of COX-11 is NS 398 which is N-(2-cyclohexyloxy-4-nitrophenyl)methanesulfonamide, commercially available from Cayman Chemical of Ann Arbor, Mich. The most preferred selective inhibitor of COX-2 for use herein is valdecoxib.

The dosage of selective inhibitor of COX-2 for the method herein is a COX-2 inhibiting amount, which is a therapeutically effective amount. The precise therapeutically effective amount of a COX-2 inhibitor to be used in the methods of the present invention can be determined by the ordinarily skilled artisan with consideration of individual differences in age, weight, and condition of the patient.

Valdecoxib belongs to a class of nonsteroidal anti-inflammatory medications (NSAIDs) called COX-2 inhibitors. Valdecoxib is available in the form of 10 and 20 mg oral, immediate release film-coated tablets by the trade name BEXTRA® (available from Pfizer® of New York, N.Y.) and Opadry® (available from Colorcon of West Point, Pa.) (Colored).

Valdecoxib is chemically designated as 4-(5-methyl-3-phenyl-4-isoxazolyl)benzene sulfonamide. Its molecular weight is 314.36 and it has an empirical formula of C₁₆H₁₄N₂O₃S. It is a second generation COX-11 inhibitor for the treatment of rheumatoid arthritis. The COX-2 enzyme plays a role in causing arthritis pain and inflammation.

Valdecoxib works by targeting the action of the COX-11 enzyme to relieve the pain, stiffness and inflammation associated with arthritis.

The following examples are provided to enable one skilled in the art to practice the invention and are merely illustrative of the invention. The examples should not be read as limiting the scope of the invention as defined in the claims.

### EXAMPLE 1

All ingredients except stearic acid were sifted through mesh # 30. The ingredients as set forth below in Table 1 were blended together by geometric dilution and mixed thoroughly in a double-cone blender and then lubricated with stearic acid that was previously passed through mesh # 60. The blend was directly compressed into tablets having target weight of about 300 mg.

TABLE 1

| Ingredient | Quantity/tab (mg) | % w/w |
|---|---|---|
| Tizanidine HCl | 6.864 | 2.29 |
| Starch 1500 | 141.04 | 47.01 |
| HPMC K100M CR | 150 | 50.00 |
| Colloidal SiO2 | 0.6 | 0.20 |
| Stearic acid | 1.5 | 0.50 |
| Average Tablet Weight (mg) | 300 | |

In Vitro Dissolution Profile

The tablets were tested in VanKel dissolution bath (USP apparatus 2, 50 rpm) at 37° C. in 500 mil of 0.01(N)HCl for 16 hours. The tizanidine in the samples was determined by an HPLC system on a C-18 column using an aqueous buffer pH 7.4: methanol with UV detection at 230 nm. The results obtained are shown in Table 2.

TABLE 2
Dissolution profile

| Time (hours) | % tizanidine release |
|---|---|
| 0 | 0 |
| 1 | 20 |
| 3 | 39 |
| 4.5 | 50 |
| 6 | 59 |
| 8 | 69 |
| 10 | 76 |
| 14 | 88 |
| 16 | 93 |

### EXAMPLE 2

The ingredients set forth below in Table 3 were sifted through mesh # 30 and mixed in a planetary mixer. The blend was then granulated using Eudragit® NE 30D dispersion (134 g of the dispersion containing 40 g of total solid content). The granules were dried to obtain a loss-on-drying (LOD) value below 2% and then milled. The granules were then passed through mesh #20 and lubricated with stearic acid in a double-cone blender. The blend was compressed into tablets having target weight of about 300 mg.

TABLE 3

| Ingredient | Quantity/tab (mg) | % w/w |
|---|---|---|
| Tizanidine HCl | 6.864 | 2.29 |
| Lactose, anhydrous | 40.64 | 13.55 |
| Starch 1500 | 61 | 20.33 |
| HPMC K100M CR | 150 | 50.00 |
| Eudragit NE 30D | 134 | 13.33 |
| | (40) | |
| Stearic acid | 1.5 | 0.50 |
| Average Tablet Weight (mg) | 300 | |

In Vitro Dissolution Profile

The tablets were then tested in a similar manner as discussed in Example 1. The results set forth below in Table 4.

TABLE 4
Dissolution profile

| Time (hours) | % tizanidine release |
|---|---|
| 0 | 0 |
| 1 | 21 |
| 3 | 42 |
| 4.5 | 52 |
| 6 | 60 |
| 8 | 69 |
| 10 | 76 |
| 14 | 85 |
| 16 | 89 |

### EXAMPLE 3

All ingredients except stearic acid were sifted through mesh # 30, mixed thoroughly in a double-cone blender and then lubricated with stearic acid that was previously passed through mesh #60. The blend was directly compressed into tablets having a target weight of about 300 mg as set forth below in Table 5.

TABLE 5

| Ingredient | Quantity/tab (mg) | % w/w |
|---|---|---|
| Tizanidine HCl | 6.864 | 2.29 |
| Lactose, anhydrous | 30 | 10.00 |
| Starch 1500 | 81.04 | 27.01 |
| HPMC K100M CR | 180 | 60.00 |
| Colloidal SiO2 | 0.6 | 0.20 |
| Stearic acid | 1.5 | 0.50 |
| Average Tablet Weight (mg) | 300 | |

In Vitro Dissolution Profile

The tablets were then tested in a similar manner as discussed in Example 1. The results set forth below in Table 6.

TABLE 6
Dissolution profile

| Time (hours) | % tizanidine release |
|---|---|
| 0 | 0 |
| 1 | 22 |
| 3 | 42 |
| 4.5 | 54 |
| 6 | 63 |
| 8 | 74 |
| 10 | 82 |
| 14 | 94 |
| 16 | 99 |

### EXAMPLE 4

Tizanidine HCl and 5% w/w of Ethocel® were sieved through mesh # 40 and dissolved with stirring in ethanol 95% to give a slightly gel-like mass. The remaining amount of Ethocel® was added to a planetary mixer along with starch 1500 and about 30% w/w of HPMC K100M and dry-mixed for 5 minutes and then granulated with the gel-like mass of tizanidine HCl and Ethocel® obtained earlier. The granules were dried to obtain a loss-on-drying value below 2%, milled and passed through mesh # 30. These granules were then blended with the remaining (20% w/w) of HPMC K100M and colloidal SiO₂ in a double-cone blender and lubricated with stearic acid that was previously passed through mesh # 60. The final blend was compressed into tablets having target weight of about 300 mg as set forth below in Table 7.

TABLE 7

| Ingredient | Quantity/tab (mg) | % w/w |
|---|---|---|
| Tizanidine HCl | 6.864 | 2.29 |
| Starch 1500 | 81.04 | 27.01 |
| HPMC K100M CR | 150 | 50.00 |
| Ethyl cellulose STD FP 100 | 60 | 20.00 |
| (Ethocel) | | |
| Colloidal SiO2 | 0.6 | 0.20 |
| Stearic acid | 1.5 | 0.50 |
| Average Tablet Weight (mg) | 300 | |

In Vitro Dissolution Profile

The tablets were then tested in a similar manner as discussed in Example 1. The results set forth below in Table 8.

TABLE 8
Dissolution profile

| Time (hours) | % tizanidine release |
|---|---|
| 0 | 0 |
| 1 | 14 |
| 3 | 30 |
| 4.5 | 38 |
| 6 | 45 |
| 8 | 53 |
| 10 | 60 |
| 14 | 69 |
| 16 | 74 |

These tablets may then be coated with an immediate release coating of valdecoxib or can be formulated into bilayer tablets containing one layer of tizanidine in sustained release matrix and a second immediate-release layer of valdecoxib. The following examples are given.

### EXAMPLE 5

### Immediate-Release Coating of Valdecoxib

The composition and method for the drug coating is as follows:

Valdecoxib: Opadry® (Colored): PEG 8000 in a proportion of 2:1:0.5 was dispersed uniformly in Methylene Dichloride: Isopropyl alcohol (40:60) solvent mixture. This dispersion was sprayed onto the tablets prepared in accordance with Examples 1-4 such that there was about 20 mg Valdecoxib in the final weight gain by each tablet.

### EXAMPLE 6

### Immediate-Release Layer of Valdecoxib

The following composition can be used to formulate Valdecoxib as immediate release granules that can be compressed with the final blend of tizanidine hydrochloride to give bilayer tablets. Conventional methods of wet granulation using water was used to formulate these granules.

TABLE 9

| Ingredient | Quantity/tab (mg) |
|---|---|
| Valdecoxib | 20 |
| Lactose monohydrate | 38.25 |
| Microcrystalline cellulose | 27 |
| Pregel starch | 5 |
| PEG 8000 | 4 |
| Croscarmellose sodium | 5 |
| FDC Yellow no. 6 (Sunset Yellow) | 0.5 |
| Magnesium stearate | 0.25 |
| Average weight of Layer (mg) | 100 |

### EXAMPLE 7

### Human PK Study of Tizanidine HCl Extended Release & Valdecoxib IR Tablets

A product of the present invention comprising Tizanidine 6 mg Extended Release formulation (as per Example 1) and Valdecoxib 20 mg Immediate Release formulation (as per Example 6) has been studied for human bioavailability in ten healthy human subjects in an open label, crossover design comparing with Tizanidine 2 mg IR tablets (Glenmark Pharmaceuticals of Mumbai, India) t.i.d. and Valdecoxib 20 mg IR tablets (Bextra®).

Pharmacokinetic Evaluation: Plasma concentrations and pharmacokinetic parameters were summarized by treatment using summary statistics. A parametric, normal-theory general linear model was applied to the untransformed and log-transformed Cₘₐₓ, C^{∞}_{avg},AUC₍₀₋ₜ₎, and AUC_{(0-∞)} parameter values.

Tizanidine Extended Release formulation has shown relative bioavailability of 63% (as reflected by AUC_{(0-∞)}, Area Under Plasma Concentration vs. time curve) with sustained levels of drug appearing up to 24 hours. A shift in peak time from 1.40 hours (for the Tizanidine IR product) to 4.10 hours (for the Tizanidine Extended Release product) and a two hour extension in half-life with respect to the Tizanidine IR product were supportive of sustained drug release from the product of present invention without any signs of dose dumping. In addition, the extended release formulation has shown Mean Residence Time (MRT) of 8.89 hours vs. 2.84 hours of the Tizanidine IR product reflecting the continuous presence of active drug levels during the dosage period.

TABLE 10
Summary PK of Tizanidine (n = 10)

| | Cₘₐₓ | C^{∞}_{avg} | AUC₍₀₋ₜ₎ | AUC_{(0-∞)} | | |
|---|---|---|---|---|---|---|
| Mean Data | (ng/ml) | (ng/ml) | (ng · hr/ml) | (ng · hr/ml) | t_{1/2} (hr) | Tₘₐₓ (hr) |
| Reference (IR | 5.45 | 1.06 | 41.25 | 42.95 | 2.25 | 1.40 |
| Product) | | | | | | |
| ER Formulation | 3.28 | 1.40 | 24.85 | 26.79 | 4.15 | 4.10 |
| Ratio (%) | 60.18 | 132.08 | 60.24 | 62.37 | 184.44 | 292.86 |

In considering the above single day data for both the Tizanidine IR & Extended Release formulations, the predicted PK parameters at doses given for 5 day chronic treatment (i.e., for the Tizanidine IR formulation the dose design was 2 mg t.i.d. for 5 days and for Extended Release formulation the dose design was 6 mg o.d. for 5 days) are set forth below in Table 11. The Extended Release formulation was found to be 100% equivalent with respect to the Tizanidine IR product.

TABLE 11
Predicted Summary PK of Tizanidine (n = 10)
after 5 day chronic treatment

| | C^{∞}_{avg} | AUC₍₀₋ₜ₎ | AUC_{(0-∞)} | | MRT |
|---|---|---|---|---|---|
| Mean Data | (ng/ml) | (ng · hr/ml) | (ng · hr/ml) | t_{1/2} (hr) | (hr) |
| Reference (IR | 1.06 | 130.51 | 130.73 | 2.25 | 2.84 |
| Product) | | | | | |
| ER | 1.08 | 128.29 | 129.49 | 4.15 | 8.89 |
| Formulation | | | | | |
| Ratio (%) | 101.89 | 98.30 | 99.05 | 184.14 | 313.03 |

TABLE 12
SUMMARY STATISTICAL EVALUATIONS OF
PHARMACOKINETIC PARAMETERS OF VALDECOXIB
UNTRANSFORMED DATA

| | | LEAST SQUARE MEANS | | | | 90% | |
|---|---|---|---|---|---|---|---|
| | | Reference | | | | CONFIDENCE | |
| | | (Glenmark | Test | RATIO | % intra | INTERVALS | |
| PARAMETER | UNITS | Formulation) | (Bextra ®) | (%) | CV | Lower | Upper |
| Cₘₐₓ | (μg · hr/ml) | 0.451 | 0.415 | 92.13 | 10.35 | 84.27 | 100.00 |
| AUC₍₀₋ₜ₎ | μg · hr/ml) | 6.497 | 6.368 | 98.02 | 8.67 | 92.24 | 103.81 |
| AUC_{(0-∞)} | (μg · hr/ml) | 7.516 | 7.340 | 97.66 | 9.33 | 91.50 | 103.82 |
| t_{1/2} | (hr) | 10.926 | 11.331 | 103.71 | | 97.12 | 110.29 |
| Tₘₐₓ | (hr) | 3.899 | 3.964 | 101.68 | | 86.40 | 116.96 |

LN-TRANSFORMED DATA

| | | | | | | 90% | |
|---|---|---|---|---|---|---|---|
| | | | | | | CONFIDENCE | |
| | | LEAST SQUARE MEANS | | RATIO | % intra | INTERVALS | |
| PARAMETER | UNITS | Reference | Test | (%) | CV | Lower | Upper |
| Cₘₐₓ | (μg · hr/ml) | 0.440 | 0.412 | 93.60 | 11.58 | 86.15 | 101.69 |
| AUC₍₀₋ₜ₎ | μg · hr/ml) | 6.404 | 6.296 | 98.32 | 8.92 | 92.70 | 104.28 |
| AUC_{(0-∞)} | (μg · hr/ml) | 7.404 | 7.219 | 97.50 | 9.61 | 91.59 | 103.80 |
| t_{1/2} | (hr) | 10.660 | 11.128 | 104.38 | | 96.92 | 112.42 |
| Tₘₐₓ | (hr) | 3.795 | 3.877 | 102.15 | | 88.97 | 117.30 |

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. A pharmaceutical formulation comprising:
a first composition comprising a therapeutically effective amount of at least one muscle relaxant; and,
a different second composition comprising a therapeutically effective amount of at least one cyclooxygenase-2 (COX II) inhibitor,
wherein the first composition provides a controlled and/or extended release of the muscle relaxant and the second composition provides a rapid and/or immediate release of the COX II inhibitor.

2. The pharmaceutical formulation of claim 1, wherein the first composition comprises
(a) a core region comprising an effective amount of the muscle relaxant and
(b) a membrane layer that extends the release of said muscle relaxant, wherein said membrane layer is selected from:
(i) a controlled release coating on said core region;
(ii) a matrix layer, which is integrated with said core region; or
(iii) a combination thereof.

3. The pharmaceutical formulation of claim 1, which is selected from the group consisting of capsules containing immediate and sustained release granules, capsules containing sustained release granules and one or more immediate release tablets, capsules containing sustained release granules and powder, extended release film or multi-layer coated tablets.

4. The pharmaceutical formulation of claim 1, wherein each of the first and second composition further comprises at least one pharmaceutical excipient.

5. The pharmaceutical formulation of claim 1, wherein the first and second compositions are in layered arrangement with respect to one another.

6. The pharmaceutical formulation of claim 3, wherein the second composition surrounds the first drug composition.

7. The pharmaceutical formulation of claim 6, wherein the first composition is in contact with the second drug composition.

8. The pharmaceutical formulation of claim 6, wherein the first drug composition is spaced-away from the second drug composition.

9. The pharmaceutical formulation of claim 1, wherein the first composition is included in a core and the second composition is included in a coat, of one or more coats, surrounding the core.

10. The pharmaceutical formulation of claim 1, wherein the first composition is a granulation, the second drug composition is a powder, granulation or compressed tablet and the formulation is a capsule.

11. The pharmaceutical formulation of claim 1, wherein said muscle relaxant is a alpha-2 adrenergic agonist.

12. The pharmaceutical formulation of claim 11, wherein the alpha-2 adrenergic agonist is tizanidine and pharmaceutically acceptable salts, isomers, and derivatives thereof.

13. The pharmaceutical formulation of claim 2, wherein said controlled release coating is comprised of a material selected from the group consisting of a water insoluble wax, a water insoluble cellulose, a water insoluble polymer, and combinations thereof.

14. The pharmaceutical formulation of claim 13, wherein said water insoluble cellulose is ethyl cellulose.

15. The pharmaceutical formulation of claim 13, wherein said water insoluble polymer is comprised of an acrylic resin.

16. The pharmaceutical formulation of claim 15, wherein said acrylic resin is poly(meth)acrylate.

17. The pharmaceutical formulation of claim 13, wherein said controlled release coating is further comprised of a water soluble polymer.

18. The pharmaceutical formulation of claim 17, wherein said water soluble polymer is polyvinyl pyrroldine.

19. The pharmaceutical formulation of claim 1, wherein said controlled release coating is further comprised of a water soluble cellulose.

20. The pharmaceutical formulation of claim 19, wherein said water soluble cellulose is hydroxypropyl methylcellulose.

21. The pharmaceutical formulation of claim 19, wherein said water soluble cellulose is hydroxypropyl cellulose.

22. The pharmaceutical formulation of claim 13, wherein said controlled release coating is further comprised of a hydrophilic pore former.

23. The pharmaceutical formulation of claim 22, wherein said hydrophilic pore former is sodium chloride.

24. The pharmaceutical formulation of claim 22, wherein said hydrophilic pore former is mannitol.

25. The pharmaceutical formulation of claim 13, wherein said controlled release coating is further comprised of a plasticizer.

26. The pharmaceutical formulation of claim 1, further comprising at least one excipient.

27. The pharmaceutical formulation of claim 2, wherein said matrix layer is selected from the group consisting of a hydrophilic polymer, a hydrophobic polymer, a hydrophobic wax, a hydrophobic fat, a hydrophobic long-chain fatty acid, a hydrophobic fatty alcohol, esters thereof, ethers thereof and mixtures thereof.

28. The pharmaceutical formulation of claim 27, wherein said hydrophilic polymer is cellulose ether.

29. The pharmaceutical formulation of claim 28, wherein said hydrophilic polymer is cellulose ester.

30. The pharmaceutical formulation of claim 28, wherein said hydrophilic polymer is an acrylic resin.

31. The pharmaceutical formulation of claim 28, wherein said hydrophilic polymer is ethyl cellulose or a salt, amide or ester thereof.

32. The pharmaceutical formulation of claim 28, wherein said hydrophilic polymer is hydroxypropyl methylcellulose or a salt, amide or ester thereof.

33. The pharmaceutical formulation of claim 28, wherein said hydrophilic polymer is hydroxypropylcellulose or a salt, amide or ester thereof.

34. The pharmaceutical formulation of claim 28, wherein said hydrophilic polymer is hydroxymethylcellulose or a salt, amide or ester thereof.

35. The pharmaceutical formulation of claim 28, wherein said hydrophilic polymer is poly(meth)acrylic acid or a salt, amide or ester thereof.

36. The pharmaceutical formulation claim 28, further comprising an excipient selected from the group consisting of a diluent, a retardant, a lubricant, a glidant and mixtures thereof.

37. The pharmaceutical formulation of claim 36, wherein said excipient is microcrystalline cellulose.

38. The pharmaceutical formulation of claim 37, wherein said microcrystalline cellulose is Avicel pH-102.

39. The pharmaceutical formulation of claim 38, wherein the concentration of said Avicel pH-102 is about 20 to about 90 percent.

40. The pharmaceutical formulation of claim 36, wherein said diluent is lactose.

41. The pharmaceutical formulation of claim 40, wherein said lactose is selected from the group consisting of hydrous lactose, anhydrous lactose, crystalline lactose, powdered lactose and mixtures thereof.

42. The pharmaceutical formulation of claim 41, wherein said lactose is direct compression grade.

43. The pharmaceutical formulation of claim 42, wherein said direct compression grade lactose is comprised of alpha lactose monohydrate and amorphous lactose.

44. The pharmaceutical formulation as of claim 37, wherein said excipient is anhydrous dibasic calcium phosphate.

45. The pharmaceutical formulation of claim 44, wherein said excipient further includes a lubricant selected from the group consisting of magnesium stearate, sodium stearyl fumarate, stearic acid and mixtures thereof.

46. The pharmaceutical formulation of claim 1, wherein said matrix layer is further comprised of pregelatinized starch.

47. The pharmaceutical formulation of claim 37, wherein said retardant is a methacrylic acid copolymer.

48. The pharmaceutical formulation of claim 47, wherein said methacrylic acid copolymer is a polymethacrylate copolymer.

49. The pharmaceutical formulation of claim 47, wherein said methacrylic acid copolymer is Eudragit RS PO.

50. The pharmaceutical formulation of claim 47, wherein said methacrylic acid copolymer is Eudragit NE 30D.

51. The pharmaceutical formulation of claim 49, wherein from about five to about twenty percent of said polymethacrylate copolymer is present in said matrix as dry powder is added to said matrix.

52. The pharmaceutical formulation of claim 27, wherein said excipient is an ethyl cellulose dispersion.

53. The pharmaceutical formulation of claim 52, wherein said ethyl cellulose dispersion is present in said matrix at a concentration of about three to about twenty percent.

54. The pharmaceutical formulation of claim 37, wherein said glidant is colloidal silicon dioxide.

55. The pharmaceutical formulation of claim 1, wherein said COX II inhibitor is selected from the group consisting of valdecoxib, celicoxib, paracoxib, etoricoxib, MK-0966, NS 398 and mixtures thereof.

56. The pharmaceutical formulation of claim 1, wherein said muscle relaxant is tizanidine and pharmaceutically acceptable salts, isomers, and derivatives thereof and said COX II inhibitor is valdecoxib.

57. A method for the treatment or prevention of pain and/or spasticity comprising administering to a subject in need of such treatment or prevention a pharmaceutically effective amount of the pharmaceutical formulation of claim 1.

58. A method for the treatment or prevention of pain and/or spasticity comprising administering to a subject in need of such treatment or prevention a pharmaceutically effective amount of the formulation of claim 5.

59. A method for the treatment or prevention of pain and/or spasticity comprising administering to a subject in need of such treatment or prevention a pharmaceutically effective amount of the formulation of claim 55.

60. A method for the treatment or prevention of pain and/or spasticity comprising administering to a subject in need of such treatment or prevention a pharmaceutically effective amount of the formulation of claim 56.

61. An orally administrable dosage form containing the pharmaceutical formulation of claim 1, wherein said dosage form provides once daily dosing for therapeutic relief from skeletal muscle spasms.

62. The dosage form of claim 61, wherein said muscle relaxant is tizanidine and pharmaceutically acceptable salts, isomers, and derivatives thereof.

63. The dosage form of claim 61, wherein said COX II inhibitor is valdecoxib.

64. The dosage form of claim 61, wherein said muscle relaxant is tizanidine and pharmaceutically acceptable salts, isomers, and derivatives thereof and said COX II inhibitor is valdecoxib.
